(19) United States
(12) Patent Application Publication
(10) Pub. No.: US 2011/0313360 A1
(43) Pub. Date: Dec. 22, 2011

(54) PEG HOLDER

(76) Inventor: Li-Ying LIN, Taipei City (TW)

(21) Appl. No.: 12/817,650

(22) Filed: Jun. 17, 2010

(51) Int. Cl. A61M 39/00

(52) U.S. Cl. 604/179

(57) ABSTRACT

A percutaneous endoscopic gastrostomy (PEG) holder for use by a patient subjected to a PEG surgery is provided. The holder is normally worn on the waist of the patient and forms an overall appearance of a waist belt, and is capable of fixing and receiving a feeding tube exposed out of the abdomen (close to stomach) of the patient after the surgery, so as to prevent the feeding tube from detaching when the patient drags the feeding tube due to carelessness.

### BACKGROUND OF THE INVENTION

1. Field of the Invention

The present invention relates to a percutaneous endoscopic gastrostomy (PEG) holder capable of being worn on the abdomen of a human body, and more particularly to a PEG holder capable of effectively fixing and receiving a feeding tube.

2. Related Art

For a patient who is unable to ingest food orally, nasogastric tube insertion generally needs to be used to administer food to the stomach of the patient with a feeding tube. However, for patients requiring long-term nasogastric tube feeding, besides unpleasant appearance, complications such as gastric hemorrhage, esophageal hemorrhage, and esophagitis caused by gastroesophageal reflux, are easily caused. For this reason, European and American countries have developed a surgery for patients in need of long-term use of a feeding tube, which is called percutaneous endoscopic gastrostomy (PEG). FIG. 1 is a schematic view of a completed PEG surgery. Referring to FIG. 1, at the abdomen 10 of a patient, an opening 11 extending into the stomach is created, and then, a feeding tube 12 is directly inserted from the abdomen 10 into the stomach 13 for feeding. It can be seen from the figure that, after inserted into the stomach 13, the feeding tube 12 is fixed to the surface of the abdomen 10 and the inner wall of the stomach 13 by using two fixers (121, 121′) disposed on the tube body, respectively, such that the feeding tube 12 is normally fixed to the abdomen 10 of the patient. Referring to FIG. 1 again, an intake port 122 is provided on one end of the feeding tube 12, through which liquid foods or other nutrients can be supplied during feeding, whereby the intake port 122 is normally exposed out of the surface of the abdomen 10. In order to avoid swinging of the feeding tube 12, an air-permeable tape 14 is adhered to the fixer 121 and the surface of the abdomen 10 for fixing. However, long-term adhesion of the air-permeable tape 14 may also cause damages to the skin on the surface of the abdomen 10 and even skin ulceration, and some serious patients may drag the intake port 122 due to unconsciousness, or a caregiver may drag the feeding tube 12 due to carelessness when helping the patient move, both of which easily lead to detachment of the feeding tube 12 due to an external force, resulting in a dangerous situation.

### SUMMARY OF THE INVENTION

In view of the above problems, the present invention is mainly directed to a PEG holder capable of being worn on the abdomen of a human body, and capable of effectively preventing a feeding tube from detaching due to dragging.

In order to achieve the above objectives, according to the PEG holder of the present invention, a through-hole is formed in a plane of a belt, an intake port is passed through the through-hole, and further a bonding portion and an, opposite bonding portion on two sides of the belt are bound, such that the belt is normally fixed to the waist of the patient; and when the intake port is not in use, its tube body can be rolled up orderly, and covered completely by a cover portion, thereby effectively preventing the feeding tube exposed out of the abdomen from detaching when dragged by an external force.

### BRIEF DESCRIPTION OF THE DRAWINGS

The present invention will become more fully understood from the detailed description given herein below for illustration only, and thus are not limitative of the present invention, and wherein:

FIG. 1 is a schematic view of a completed PEG surgery;

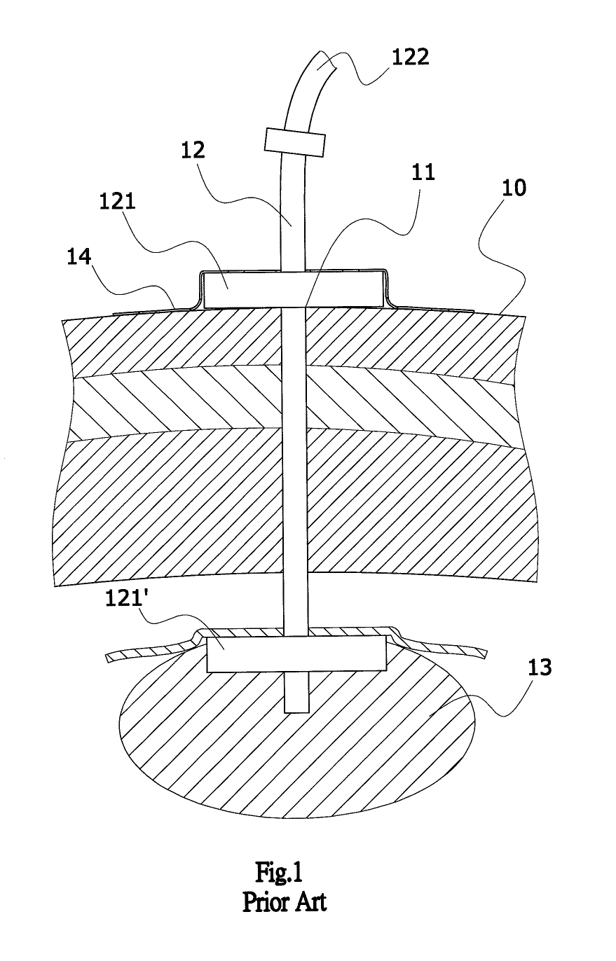

FIG. 2 is a three-dimensional schematic view of the present invention;

FIG. 3 is a schematic view (1) of the implementation of the present invention;

FIG. 4 is a schematic view (2) of the implementation of the present invention;

FIG. 5 is a schematic view (3) of the implementation of the present invention;

FIG. 6 is a schematic view (4) of the implementation of the present invention;

FIG. 7 is a schematic view of a preferred embodiment (1) of the present invention; and

FIG. 8 is a schematic view of a preferred embodiment (2) of the present invention.

### DETAILED DESCRIPTION OF THE INVENTION

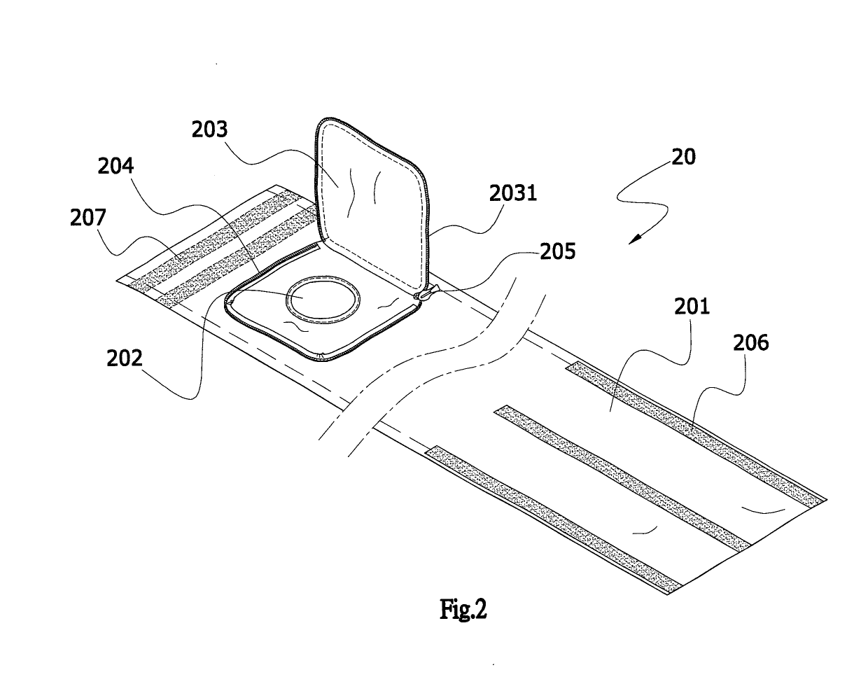

FIG. 2 is a three-dimensional schematic view of the present invention. Referring to FIG. 2, in a PEG holder 20, a through-hole 202 is formed in a plane of a belt 201. A cover portion 203 is sewn at a proper position around the through-hole 202, and the cover portion 203 can be opened or closed relative to the through-hole 202. A zipper portion 2031 is sewn on a side edge of the cover portion 203, and an opposite zipper portion 204 is sewn around the through-hole 202. The zipper portion 2031 can engage with the opposite zipper portion 204 through a zipper 205, such that the cover portion 203 and the belt 201 form a unity. Furthermore, a bonding portion 206, for example a Velcro strip, is sewn on one side of a front surface of the belt 201, and an opposite bonding portion 207, for example an opposite Velcro strip, is sewn on a back surface of the belt 201, and the bonding portion 206 and the opposite bonding portion 207 can be bound and thus fixed to each other.

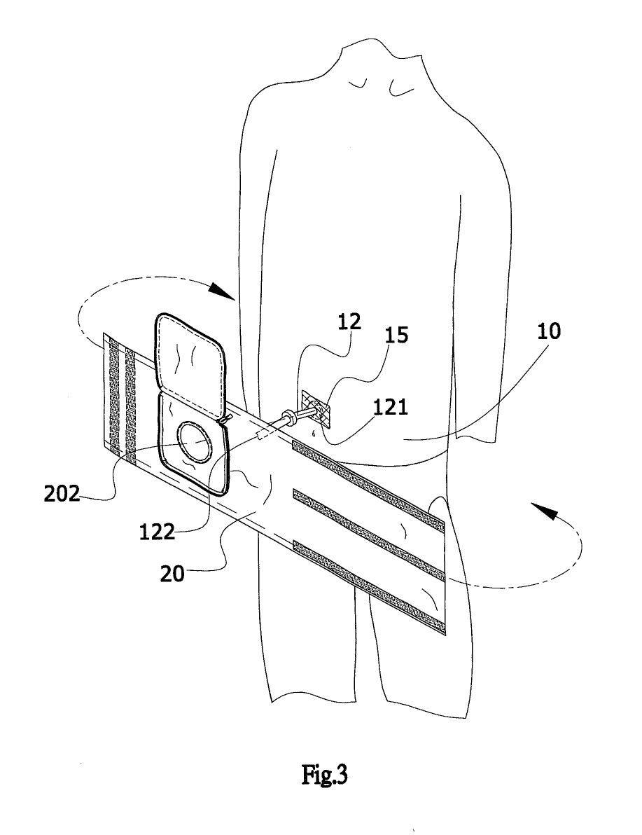

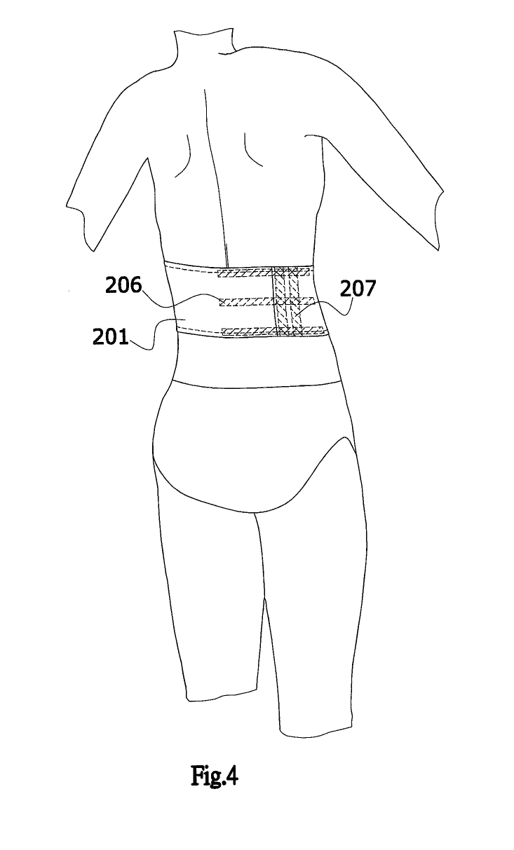

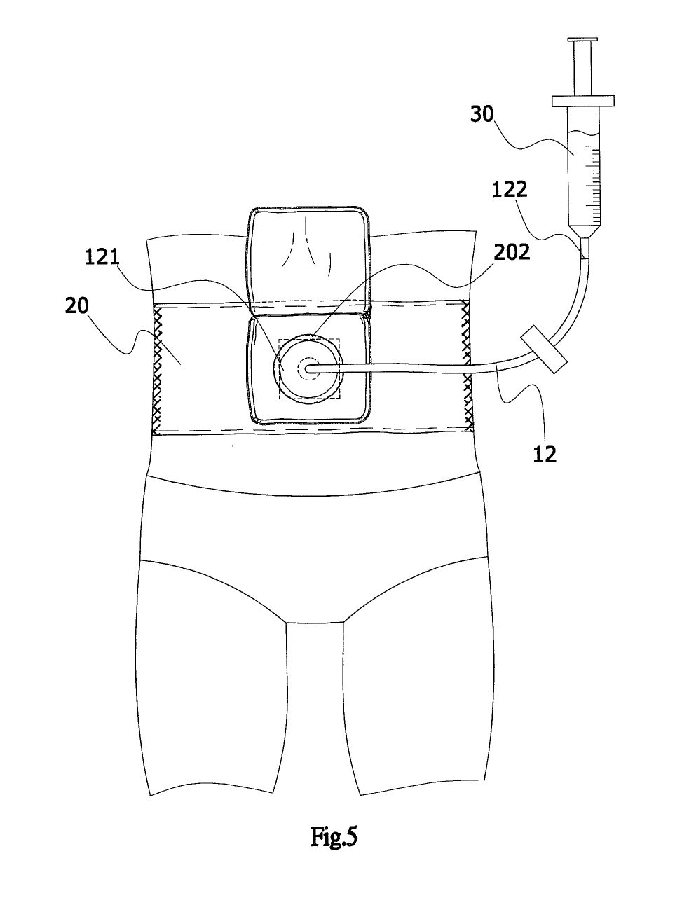

FIG. 3 is a schematic view (1) of the implementation of the present invention. Referring to FIGS. 2 and 3, after a patient has been subjected to a PEG surgery, the intake port 122 of the feeding tube 12 is normally exposed out of the abdomen 10 of the patient. Also, in the implementation of the present invention, a layer of air-permeable gauze 15 may be covered around the fixer 121 in advance, so as to maintain the skin around the opening for inserting the feeding tube 12 clean, and avoid infection. Further, the intake port 122 of the feeding tube 12 is passed through the through-hole 202 in the plane of the PEG holder 20, such that the belt 201 is parallel to and in close proximity to the surface of the abdomen 10 of the patient, and at this time, two sides of the belt 201 are wound to the back of the patient. FIG. 4 is a schematic view (2) of the implementation of the present invention. Referring to FIG. 4, based on those described in FIG. 3, when two sides of the belt 201 are wound to the back of the patient, the bonding portion 206 and the opposite bonding portion 207 on the two sides of the belt 201 overlap each other, and are further bound and thus fixed to each other. The range of adhesion of the bonding portion 206 to the opposite bonding portion 207 can be adjusted depending on the waistline of the patient, such that the present invention can be firmly secured to the waist of the patient. An aspect in which the present invention is secured to the waist of the patient is as shown in FIG. 5, which is a schematic view (3) of the implementation of the present invention. Referring to FIG. 5, the feeding tube 12 is normally exposed out of the through-hole 202 of the PEG holder 20. When the patient wants to take food, the intake port 122 is connected to an external nutrient supply device 30, for example, a feeding bag or feeding syringe, to inject the food into the intake port 122 and then the stomach of the patient. It can be seen from the above that, in the implementation of the present invention, a layer of gauze 15 is covered around the fixer 121 in advance, and then the belt 201 is worn on the abdomen of the patient, so as to completely cover the gauze 15, such that fixation is achieved without adhesion of any air-permeable tape. Thus, damages to the skin around the fixer 121 due to long-term adhesion of the air-permeable tape, such as ulceration and swelling, can be avoided.

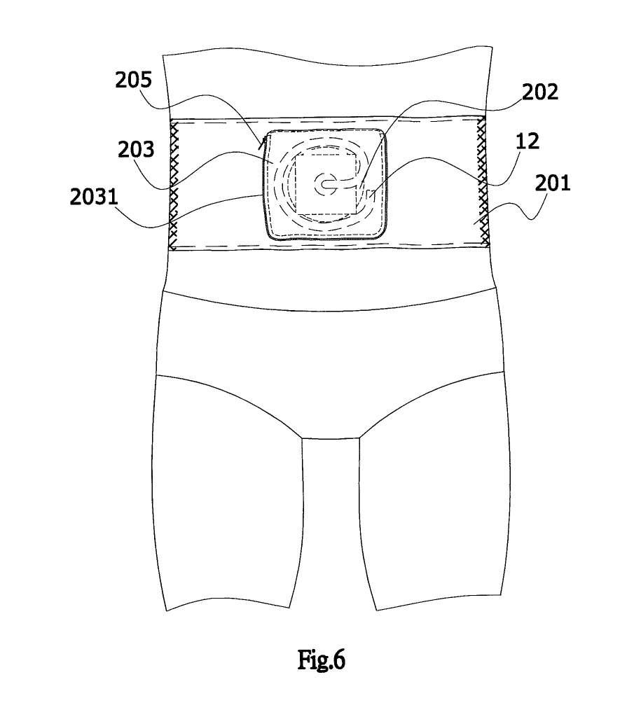

FIG. 6 is a schematic view (4) of the implementation of the present invention. Referring to FIG. 6, based on those described in FIG. 5, when the feeding tube 12 is normally not in use, its tube body can be further rolled up orderly, and placed around the through-hole 202 of the belt 201. Further, the cover portion 203 is covered on the tube body completely, and then engagement is effected through the zipper 205, such that the feeding tube 12 is completely wrapped between the cover portion 203 and the belt 201, and thus completely received, thereby effectively preventing the exposed feeding tube 12 from detaching when dragged by an external force.

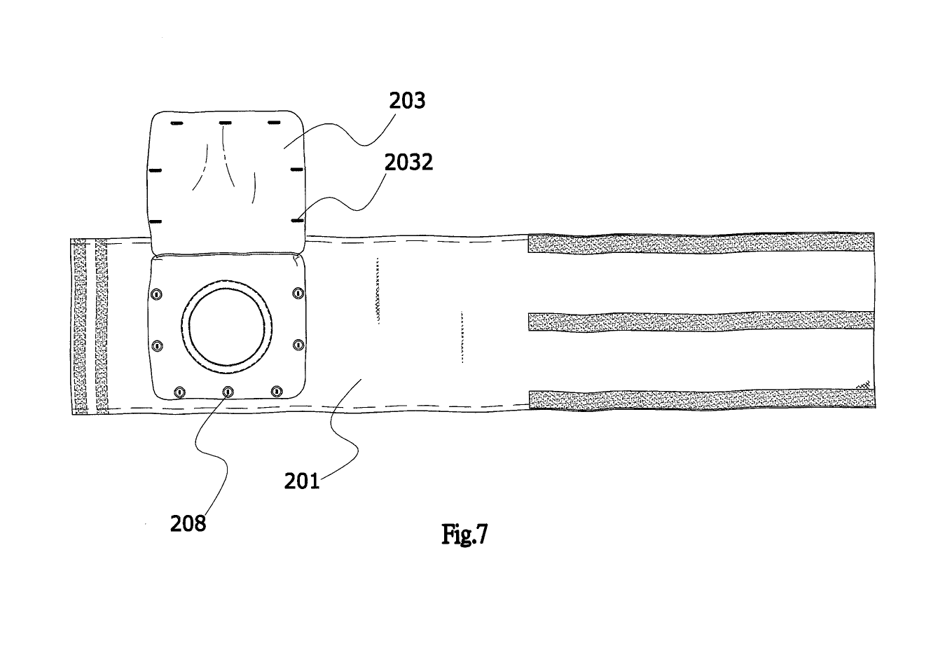

FIG. 7 is a schematic view of a preferred embodiment (1) of the present invention. Referring to FIG. 7, according to the present invention, a plurality of fastening portions 2032, for example a seam, is formed along a plane of the cover portion 203 in order, and a plurality of opposite fastening portions 208, for example a button, is sewn in the plane of the belt 201 at positions opposite to the fastening portions 2032, such that when the cover portion 203 is covered on the plane of the belt 201, the cover portion 203 can be normally fixed to the surface of the belt 201 through fastening between the fastening portions 2032 and the opposite fastening portions 208 respectively.

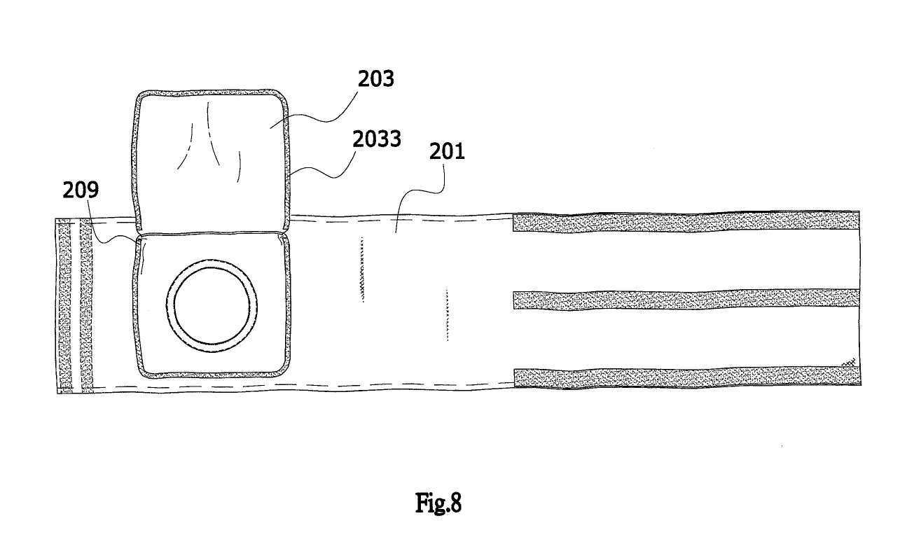

FIG. 8 is a schematic view of a preferred embodiment (2) of the present invention. Referring to FIG. 8, based on those described in FIG. 7, a Velcro strip 2033 is sewn along an outer side of the cover portion 203, and an opposite Velcro strip 209 is sewn in the plane of the belt 201 at a position opposite to the Velcro strip 2033, such that when the cover portion 203 is covered on the plane of the belt 201, the cover portion 203 can be normally adhered to the surface of the belt 201 through bonding between the Velcro strip 2033 and the opposite Velcro strip 209 respectively.

As described above, after the present invention is implemented accordingly, an accommodation space is formed between the cover portion and the surface of the belt, and the tube body of the feeding tube can be rolled up and well received in the accommodation space, and can be wrapped completely by the cover portion. Therefore, the objective of providing a PEG holder capable of effectively preventing the feeding tube from detaching due to dragging can surely be achieved.

The above descriptions are merely preferred embodiments of the present invention, but are not intended to limit the present invention. Any equivalent variation and modification made by persons skilled in the art without departing from the spirit and scope of the present invention shall fall within the appended claims of the present invention.

What is claimed is:

1. A percutaneous endoscopic gastrostomy (PEG) holder, capable of being worn on the waist of a patient, comprising:
a belt, wherein a through-hole is formed in a plane of the belt, and an opposite zipper portion is sewn around the through-hole;
a bonding portion and an opposite bonding portion, sewn on two sides of the belt, respectively; and
a cover portion, disposed at an outer periphery of the through-hole, wherein a zipper portion is sewn along a side edge of the cover portion, and the zipper portion is capable of engagement with the opposite zipper portion through a zipper.

2. The PEG holder according to claim 1, wherein the bonding portion is a Velcro strip.

3. The PEG holder according to claim 1, wherein the opposite bonding portion is an opposite Velcro strip.

4. A percutaneous endoscopic gastrostomy (PEG) holder, capable of being worn on the waist of a patient, comprising:
a belt, wherein a through-hole is formed in a plane of the belt, and a fastening portion is disposed around the through-hole;
a bonding portion and an opposite bonding portion, sewn on two sides of the belt, respectively; and
a cover portion, disposed at an outer periphery of the through-hole, wherein a plurality of opposite fastening portions is disposed around the through-hole along a side edge of the cover portion.

5. The PEG holder according to claim 4, wherein the opposite fastening portion is a button.

6. The PEG holder according to claim 4, wherein the fastening portion is a seam.

7. A percutaneous endoscopic gastrostomy (PEG) holder, capable of being worn on the waist of a patient, comprising:
a belt, wherein a through-hole is formed in a plane of the belt, and a Velcro strip is disposed around the through-hole;
a bonding portion and an opposite bonding portion, sewn on two sides of the belt, respectively; and
a cover portion, disposed at an outer periphery of the through-hole, wherein an opposite Velcro strip is disposed around the through-hole along a side edge of the cover portion.
